(19) United States
(12) Patent Application Publication
(10) Pub. No.: US 2003/0208101 A1
(43) Pub. Date: Nov. 6, 2003

(54) Embryo-implanting catheter control system and method of the same

(76) Inventor: Michael D. Cecchi, Madison, CT (US)

Correspondence Address: CUMMINGS AND LOCKWOOD, GRANITE SQUARE, 700 STATE STREET, P O BOX 1960, NEW HAVEN, CT, 06509-1960, US

(21) Appl. No.: 10/138,776

(22) Filed: May 3, 2002

(51) Int. Cl. A61B 17/43

(52) U.S. Cl. 600/34

(57) ABSTRACT

A catheter assembly for performing embryo implants in a female's uterus including an inner component having an echogenic soft distal tip for supporting the embryo to be implanted in the womb. A guide cannula receives the inner component which in combination with the rigid proximal portion facilitates insertion of the distal tip and embryo into the womb through the cervix. The guide cannula is a hollow tubular member attached to the hub, and a distal end which protects the embryo on the inner component during insertion. Markings indicate the position of the soft distal tip of the inner component with respect to the guide cannula as well as the overall length of the catheter assembly. During the implantation process, ultrasound imaging equipment is used to monitor the position of the echogenic distal tip.

### TECHNICAL FIELD

The subject disclosure relates to a control system for an embryo transfer catheter assembly, which includes an outer guide cannula, and an inner catheter, which is slidably disposed in a bore in the guide cannula. The catheter assembly is particularly useful for safely depositing an embryo on the uterine wall of a female seeking to conceive. The catheter includes a soft distal end, which carries the embryo, and a more rigid outer cannula, which protects the inner catheter during use. More particularly, the subject control system includes a marking system to identify the inner catheter relationship to the outer guide cannula and thereby insure the safety of the contained embryos. Further, the control system includes an echogenic portion to allow for recognition of the distal end of the catheter during an ultrasound.

### BACKGROUND ART

Catheters for use in penetrating various passages in the human body for various procedures are well known in the prior art. Uses for such catheters include: penetrating cardiac blood vessels; penetrating cerebral blood vessels; and penetrating the uterus in embryo implant procedures, for example. Catheters used in the aforesaid procedures all include a distal end, which penetrates the body passages, and a proximal end, which remains outside of the body and is used to manually “steer” the distal end of the catheter through the body passages. The aforesaid catheters are typically inserted into the body through the bore of a proximal catheter guide cannula, which penetrates the body. Once the guide cannula penetrates the body passage in question, the catheter is advanced into the body passage through the guide cannula.

In order to avoid damaging delicate body tissues, the distal tip of the catheter should be formed from a soft material. U.S. Pat. Nos. 4,531,943 to Van Tassel et al to 4,863,442 to DeMello et al 4,886,506 to Lovgren et al; 5,045,072 to Castillo et al 5,221,270 to Parker; 5,234,416 to Macaulay et al; 5,571,073 to Castillo; 5,769,830 to Parker; 5,792,124 to Horrigan et al.; and 6,165,165 to Cecchi et al. all describe catheters that are provided with soft distal ends so as to avoid tissue damage when inserted into the body and each is incorporated herein by reference. Each of these patents describes the need for a soft inner catheter. The prior art also recognizes the fact that while the distal end of the catheter should be soft, more proximal portions of the catheter should preferably be more rigid in order to facilitate pushing of the catheter into the body through the guide cannula.

The application of a soft inner catheter is particularly troublesome in the field of implanting an embryo into a female's womb. A portion of the problem is the result of cervical constriction, which is encountered by a physician during the implant procedure when an embryo is implanted in a female's uterus. As a result of the cervical constriction, the guide cannula will be advanced into the os of the cervix. A further problem encountered by physicians is the length of the catheters exposed distal portion and the varying length that may extend past the distal tip of the guide cannula. Lengths of the exposed proximal portion vary due to poor design and manufacturing tolerances. In particular, the manufacturer produces catheters of varying length and the change in the relationship by the physician retracting the inner catheter to the guide cannula results in an undefined length being exposed.

During insertion of the guide cannula, the embryo implant catheter will be retracted completely inside of the guide cannula so as to protect the embryo from loss or damage. Once the guide cannula is in position in the os of the cervix, the inner catheter, the distal tip of which holds the embryo to be implanted, is then advanced through the guide cannula and through the cervix and into the uterus. However, lacking a clear mark on the inner catheter, which lets the physician know that the inner catheter is safely within the outer guide, close to the tip and ready for advancement, the physician must exercise skill and judgment to successfully implant the embryo. Therefore, what is needed is a catheter assembly which lets the physician precisely monitor and control the position of the inner catheter.

An additional problem that exists is the various lengths of catheters necessary for the actual penetration of the restricted os and the safeguarding of the embryos. Typically, the embryos are located in the first 1 cm, at the distal end of the implantation catheter. As described earlier, this is accomplished by retracting the inner catheter to bring the embryos, which are in the first 1 cm to be within the guide cannula. The guide cannula is then partially advanced into the os, opening its passageway, and then the tip of the soft inner catheter is advanced through this “opened” os. The problem that remains is the lack of uncertainty that the inner catheter has remained in position during this process. If the inner catheter has moved, the physician has no way of determining and correcting the error. It is, therefore, an object of this invention to establish a marking system of clear and distinct markings on the catheter assembly that will allow the physician to clearly know the relationship of the inner catheter to the guide cannula and therefore the location of the distal tip.

Prior art catheters recognize the need for a soft inner catheter, but none address the problem of soft catheters inherent quality of flexing and buckling during insertion through the cervical os and the possible damage or loss of the embryos at that time. Also, physicians encounter difficulty when using catheters, which have various lengths of exposed distal portions of the catheter. This differential in exposed distal portion is created by the manufacturer to accommodate a range of procedural difficulties and typically ranges from 3 cm to 5 cm. Physicians must draw the distal tip safely into the outer cannula to protect the embryos from loss or damage however, with varying lengths of exposed distal portion a high skill level is not easily achieved. Therefore, what is needed is a catheter assembly which facilitates control by the physician despite variations in the lengths of the catheters.

Physicians and embryologists are also finding a growing need to track the position of the catheter tip actively by employing an ultrasonic imaging device. To be recognized during an ultrasound, the tip must be at least partially echogenic. By using an echogenic tip to retain the embryos, an accurate placement inside the uterus can be actively verified.

Typically, a catheter tip is not conducive to modification for ultrasound recognition. Other medical devices may be grooved or etched to create an echogenic surface. Another unsuitable technique has been to coat the device with an echogenic solution. The thin walls of a delicate catheter tip suffer damage or at least reduced performance characteristics by such methods such as, without limitation, reduced flexibility and increased mucous attachment. Still further, the coating solution may even be toxic to the embryonic system and detrimental to the embryo development and cell culture.

In view of the above, attempts have been made to produce echogenic catheters. For example, one catheter tip includes a 3 mm wide metal ring. The metal ring is set approximately 3 mm from the distal tip to allow approximate determination of the location of the embryos. Additional indentations on the ring may further enhance the echogenic property thereof.

There are problems associated with the metal ring. The ring collects mucous and makes the catheter more invasive which may result in damage to the inner uterine wall. Further, addition of the metal ring requires added steps and expense in the manufacturing of such a catheter.

There is a need, therefore, for an improved catheter which permits easy recognition of the location of the tip during an ultrasound and aids in assuring appropriate positioning of the inner component holding the embryo with respect to the outer guide cannula.

### SUMMARY OF THE INVENTION

The subject disclosure relates to an improved catheter assembly, which is particularly useful in performing embryo implants in a female's uterus. The catheter assembly includes an inner component having a soft distal tip for supporting the embryo to be implanted in the womb, and a more rigid proximal portion. A guide cannula receives the inner component which in combination with the rigid proximal portion facilitates insertion of the distal tip and embryo into the womb through the cervix. The guide cannula further includes a hollow rigid proximal hub, which is grasped by the physician during the implant procedure. The guide cannula has a hollow tubular member attached to the hub, and a distal end which protects the embryo during insertion.

In a preferred embodiment, the distal end of the inner component is echogenic and soft, typically having a durometer of about 80 Shore A. Preferably, the inner component is a tubular member formed from an extruded resin such as PVC or polyurethane. The guide cannula is preferably formed from co-extruded concentric selected resins, wherein the inner resin has a low coefficient of friction, i.e., a “slippery” resin, such as nylon, for example; and the outer resin has a higher coefficient of friction, and possesses memory so that the guide cannula can be easily grasped, and can be bent, and will retain its bent configuration. The catheter is made in various length configurations for the placement of embryos. For example, the overall length of the assembly may vary from 15 cm in length to 25 cm in length to accommodate the various depths and sizes of the uterus and physical size of the patient. Preferably the lengths of the exposed inner catheter in relationship to the guide cannula varies. Typically, the exposed length varies from 3 cm to 5 cm.

A 5 cm exposed length of the exposed distal portion allows full penetration of this length and embryo placement. Alternatively, a 3½ cm length of the exposed distal length is preferred when the uteral os is restricted and more difficult to penetrate. The shorter exposed distal portion allows the user to maintain better control.

It would be highly desirable to provide a separate and distinct marking on the inner catheter to ensure that the inner catheter is properly positioned within and near the end of the outer guide.

It would be highly desirable for the protection and safety of embryos to have an embryo transfer catheter which has a series of uniquely different marks to form a purposeful marking system imprinted along the catheter and outer guide cannula to indicate to the user the exact location of the inner component with respect to the distal end of the guide cannula.

It is, therefore, an object of this invention to clearly and distinctly mark the inner catheter with various and different types of marks that will allow the physician to clearly know where the distal tip of the inner catheter tip is in relation to the outer guide cannula.

It is, therefore, an object of this invention to clearly and distinctly mark the inner catheter with various and different types of marks that will allow the physician to clearly identify the relationship of the outer guide cannula when the inner catheter is retracted.

It is, therefore, an object of this invention to clearly and distinctly mark the inner catheter with various and different types of marks that will allow the physician to clearly identify the relationship of the outer guide cannula when the inner catheter is extended.

It is, therefore, an object of this invention to clearly and distinctly mark the inner catheter with various and different types of marks that will allow the physician to clearly identify the relationship of the outer guide cannula and the inner catheter during the implantation procedure.

It is another object of this invention to provide a marking system of an embryo transfer catheter having different style markings to discriminate the feature of the catheter between overall length characteristics and positioning characteristics.

It is an additional object of this invention to provide a method for marking an embryo implant catheter with different characteristic markings between overall length characteristics and positioning characteristics.

It is an additional object of this invention to provide a method for marking an embryo implant catheter with different characteristic markings between overall length characteristics and positioning characteristics by the use of various colors.

It is an additional object of this invention to provide a method for marking an embryo implant catheter with different characteristic marking between overall length characteristics and positioning characteristics by the use of various marking designs.

Thus, a clear mark on the inner catheter, which lets the physician know that the inner catheter is safely within the outer guide and close to the tip, ready for advancement.

It would be highly desirable to provide a separate and distinct marking on the inner catheter to ensure that the inner catheter is properly positioned within and near the end of the outer guide.

It would be highly desirable for the protection and safety of embryos to have an embryo transfer catheter which is uniquely different with a purposeful marking system imprinted along the catheter and outer guide cannula to indicate to the user the exact location of the inner component with respect to the distal end of the guide cannula.

It should be appreciated that the present disclosure can be implemented in numerous ways, including without limitation as a process, an apparatus, a system, a device or a method.

It would be highly desirable to provide an echogenic distal end of the inner component. Preferably, the inner component is formed from an extruded resin such as, without limitation, polyurethane.

It is, therefore, an object of this invention to be able to make an embryo transfer catheter that remains soft and safe while still detectable under ultrasound conditions in order to help the physician direct and properly place the embryos within the uterus.

It is, therefore, an object of this invention to be able to make an embryo transfer catheter that remains soft, remains less traumatic and that may be detected under ultrasound conditions to help direct and properly place the embryos without adding expenses and additional manufacturing steps.

These and other unique features of the system disclosed herein will become more readily apparent from the following description, the accompanying drawings and the appended claims.

### BRIEF DESCRIPTION OF THE DRAWINGS

FIG. 1 is a disassembled view of a catheter assembly formed in accordance with the subject disclosure.

FIG. 2 is a side elevational view of the catheter assembly of FIG. 1 assembled.

FIG. 3 is a side elevational view of the catheter assembly of FIG. 1 with the inner component retracted within the guide cannula.

FIG. 4 is a side elevational view of the catheter assembly of FIG. 1 with the inner component extending beyond the guide cannula.

FIG. 5 is an enlarged localized view of the distal tip of the catheter assembly of FIG. 1.

FIG. 6 is an enlarged localized view of the distal tip prior to insertion into the tip forming die.

FIG. 7 is an enlarged localized cross-sectional view of the distal tip being formed in the die.

### DETAILED DESCRIPTION OF SPECIFIC EMBODIMENTS OF THE INVENTION

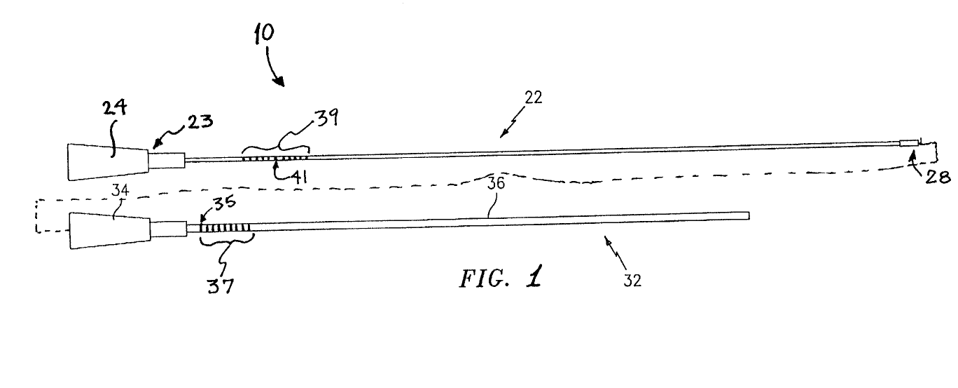

Referring to FIG. 1, the catheter assembly 10 includes a guide cannula 36 which defines an elongated channel for receiving an inner component 22. The guide cannula 32 includes a tubular hub 34 attached to the proximal end 35 of a tubular guide member 36. A series of marks 37 on the guide member 36 adjacent the hub 34 indicates the length of guide member 36 between each mark and the distal end 38 thereof. For example, the mark 37 closest to the hub 34 may indicate that when the guide cannula 32 is inserted to such mark 37, the length of the guide cannula within the patient is 22 cm. Each successive mark 37 is spaced 1 cm further out on the guide member 36. Accordingly, insertion only up to the 9th mark 37, i.e. the most distal mark, indicates that 14 cm of the guide cannula is within the patient.

The inner component 22 includes a member 26 attached to a hub 24 at a proximal end 23. A distal end 28 is configured to receive and retain an embryo. Preferably, a portion of the distal end 28 is echogenic. Markings 39 provide visual reference points for locating the embryo on the distal end 28 with respect to the distal end 38 of the guide cannula 32 when the inner component 22 is within the guide member 36. Preferably, a central marking 41 indicates the distal end 28 of the inner component 22 and the distal end 38 of the guide cannula 32 are flush. Each marking 39 out from central marking 41 indicates movement of 1 cm of the inner component 22 with respect to the guide cannula 32. Consequently, when the inner component 22 extends outside of the guide cannula 32, the length of the catheter assembly 10 inserted within the patient is determined by adding the readings of inner component markings 39 and the guide cannula markings 37. In one embodiment, markings 39 are black bands and central marking 41 is a red band. In alternative embodiments, the markings 39 are x-shaped, varying widths and combinations thereof. It is envisioned that markings 39 and 37 may vary in shape, size or color.

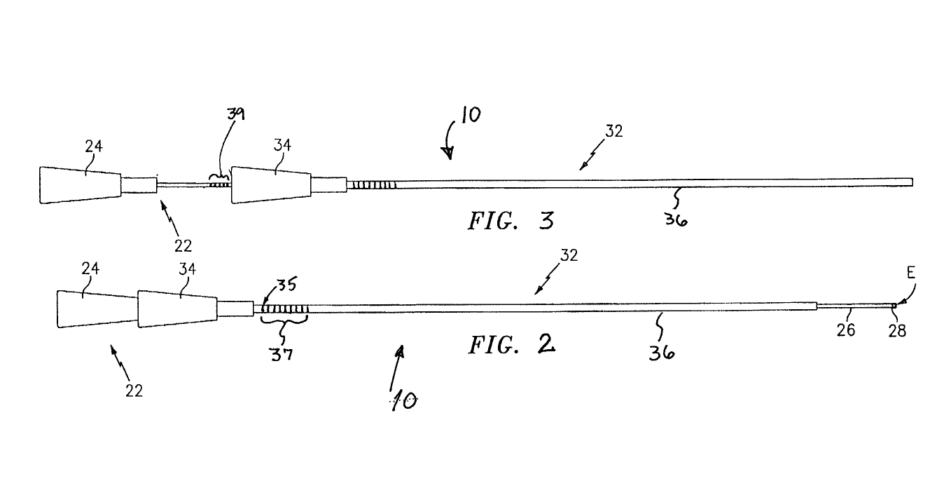

Referring to FIG. 2, in operation, the inner component 22 is inserted into the tubular guide member 36 until hub 24 is seated within the hub 34. Preferably the seating uses a friction lock inside such as a graduated luer lock as is well known to those of ordinary skill in the pertinent art. As a result, the distal portion 28 of the inner component 22 projects beyond the guide cannula 32 and is exposed. The embryo(s) are loaded into the distal end 28 of the inner component 22 and held, by suction or other well known conventional means.

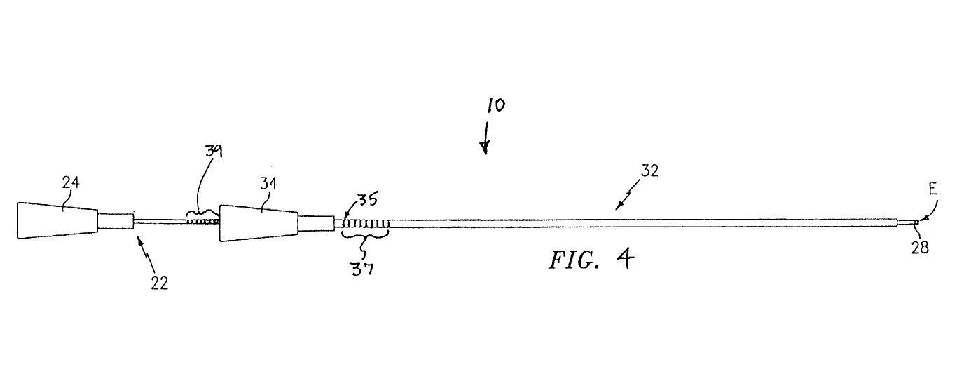

Referring now to FIG. 3, upon loading the embryo, the inner component 22 is retracted within the tubular guide member 36 which guides and protects the distal end 28 during insertion. Preferably, the distal end 28 of the component 22 is near the distal end 38 of the guide cannula 32 and protected by the tubular guide member 36 and does not extend beyond the guide cannula distal end 38. When the component is retracted until central marking 41 can be seen, the physician can be certain the embryo is just inside the tubular guide member 36. The adjacent markings 39 provide reference to allow the physician to select a desired depth to which the distal end 28 of the inner component is retracted within the tubular guide member 36. In certain alternative cases, the catheter assembly 10 may be inserted into the uterus in a position with the distal end 28 exposed. In such cases, the physician uses the adjacent markings 39 to select the length of distal end 28 which is exposed. In short, the exposed distal portion 28 varies by the relationship in the length of inner component 22 to the length of the guide cannula 32, and is defined by markings 39 in advance according to the desired use and expected ease or difficulty of the embryo transfer. Upon setting the inner component 22 to the desired position, the catheter assembly is inserted. The depth of insertion can be determined by the markings 37 on the guide cannula 32.

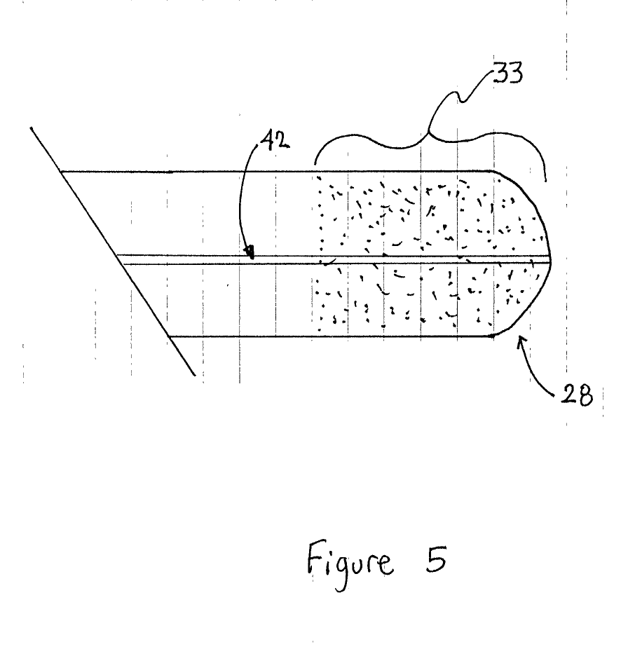

Referring now to FIG. 4, when catheter assembly 10 is advanced into the uteral os the desired depth, a partial opening of os of the cervix occurs. Upon opening the os, the inner component 22 is advanced within the guide cannula 32 until the distal end 28 extends a desired distance into the uterus to expose the distal end 28 and to allow the implant of an embryo into the uterus. Markings 39 indicate to the physician the length of the exposed distal end 28 of the inner component.

In another embodiment, a catheter assembly marking system has various separate and distinct markings along the inner component 22 and the guide cannula 32. Each marking is unique and distinct in shape, size or color according to the specific purpose and need and will allow the user to select placement according to preventing loss or damage to the embryo during the procedure. The resulting marks allow the user to accurately and safely retract the embryos held in the distal end portion within the guide cannula during the insertion into the uterus os.

It is readily appreciated that an improved catheter assembly 10 is provided by being able to accurately retract the inner component 22 within the more rigid guide cannula 32. The improved catheter assembly 10 is obtained without the need to assemble separate catheter tube components. Further, the catheter assembly 10 is easy to use by one person and the markings 37 and 39 are highly visible to the user.

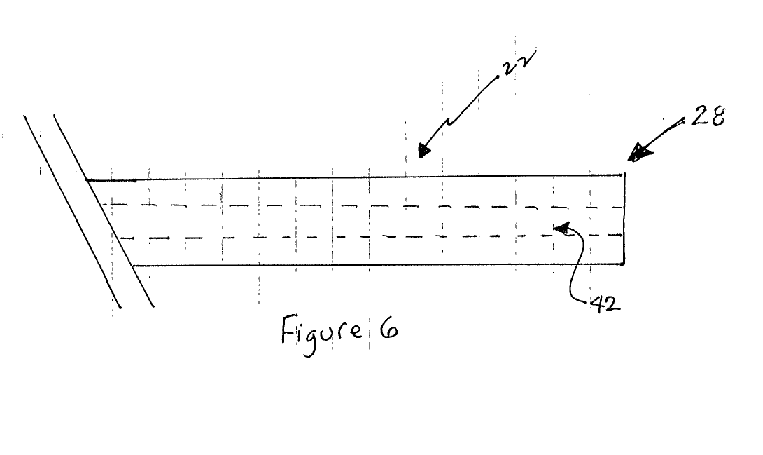

Referring to FIG. 5, in a preferred embodiment, to further aid the physician, the distal end 28 is echogenic. Accordingly, during insertion of the catheter assembly 10, the location of the distal end, i.e. the embryo, with respect to the patient can be tracked via ultrasound imaging equipment.

Preferably, the echogenic portion 33 of the distal end 28 is approximately 1 cm, although variations between 0.2 and 2 cm in length would accommodate most applications. The echogenic portion 33 can be seen by ultrasonic imaging equipment because the distal end 28 of the inner component 22 has been compressed to a greater density than the remainder of the inner component 22 as will be described hereinbelow. In another embodiment, the thickness of the echogenic portion 33 is increased to make the echogenic portion 33 opaque to ultrasonic imaging. In another embodiment, the echogenic portion 33 is a greater density and thickness. It is also envisioned that the entire inner component 22 may be echogenic. Preferably, the inner component 22 has an open central passage 42 (shown in shadow lines) even though the distal end 28 has been compressed.

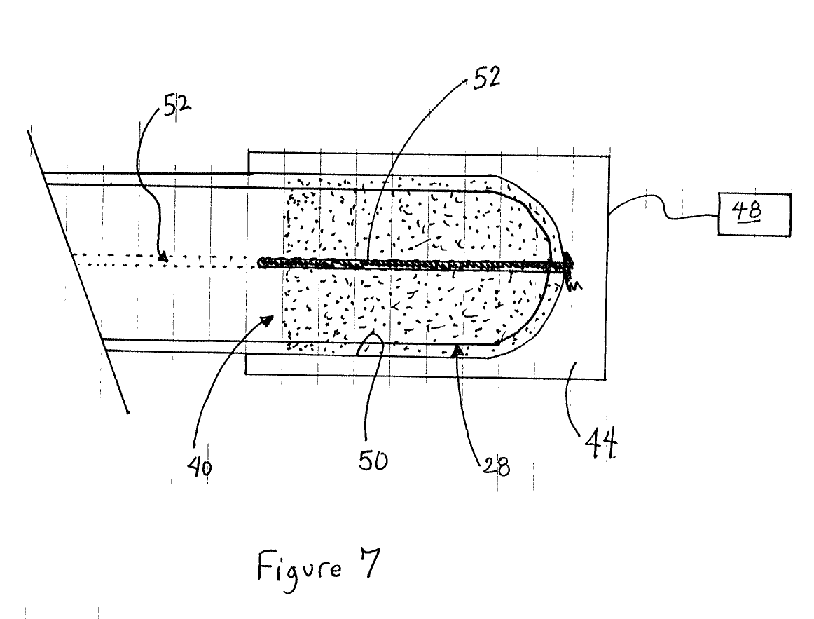

Referring now to FIGS. 6 and 7, the process for forming the distal end 28 with an echogenic portion 33 is described. As best seen in FIG. 6, prior to forming, the distal end 28 of the inner component 22 is a bluntly cut end with a centrally located passage 42. Typically, the distal end 28 is formed by extrusion as is well known to those of ordinary skill in the pertinent art.

To increase the density and thereby the echogenic properties of the distal end 28, the blunt distal end 28 is formed in an elongated die 44. The elongated die 44 has a cavity 46 for receiving the distal end 28. A heat source 48 is provided to heat an inner surface 50 of the cavity 46 prior to the forming operation. A center post 52 extends from the closed end of the cavity 46 for insuring the central passage 42 remains open during and after forming. When the inner surface 50 of the cavity 46 reaches the desired temperature, the central passage 42 of the blunt distal end 28 is positioned about the center post 52 and the inner component 22 is forced towards the closed end of the elongated die 44. The compressive force against the heated inner surface 50 of the cavity 46 reshapes the distal end 28 and compresses the material thereof. Consequently, the distal end 28 is integrally formed and echogenic. After forming the distal end 28, an embryo holding component or other desired modifications can be performed to customize the distal end 28 for the desired application.

While the invention has been described with respect to preferred embodiments, those skilled in the art will readily appreciate that various changes and/or modifications can be made to the invention without departing from the spirit or scope of the invention as defined by the appended claims.

What is claimed is:

1. A catheter assembly for protecting an embryo during implantation, comprising:
an elongated component having a proximal end and a distal end, the distal end for receiving and retaining an embryo, the elongated component further including an intermediate portion between the proximal end and the distal end, the intermediate portion having at least one mark; and
a guide cannula defining a channel for receiving the elongated component, the guide cannula having a guide member distal portion and a proximal portion, wherein the at least one mark indicates a position of the distal end of the elongated component with respect to the guide member distal portion when the elongated component is inserted therein.

2. A catheter assembly as recited in claim 1, wherein the at least one mark indicates the distal end of the component is fully retracted within the guide member distal portion.

3. A catheter assembly as recited in claim 1, wherein the at least one mark is a series of ruled marks.

4. A catheter assembly as recited in claim 3, wherein one of the series of ruled marks is larger and a different color to indicate a retracted position.

5. A catheter assembly as recited in claim 3, wherein one of the series of ruled marks is larger and a different color to indicate an extended position.

6. A catheter assembly as recited in claim 1, wherein the distal end of the elongated component is at least partially echogenic.

7. An embryo implant catheter assembly comprising:
first means for receiving an embryo;
second means for receiving the first means in variable positions, the variable positions including a retracted position for facilitating insertion of the first means and an extended position for implanting the embryo; and
third means for indicating the retracted position and the extended position, wherein the means for indicating is associated with at least one of the first means and the second means.

8. An embryo implant catheter assembly as recited in claim 7, wherein the third means also indicates intermediate positions between the retracted and extended positions.

9. An embryo implant catheter assembly as recited in claim 7, wherein the first means is an elongated member attached to a hub at a proximal end and having a distal end for receiving the embryo.

10. An embryo implant catheter assembly as recited in claim 7, wherein the second means is a tubular guide cannula having a hub attached to a proximal end thereof.

11. An embryo implant catheter assembly as recited in claim 7, wherein the third means for indicating is a colored line on the first means.

12. An embryo implant catheter assembly as recited in claim 7, wherein the first means is at least partially echogenic.

13. A catheter assembly for implanting an embryo during an ultrasound imaging process, comprising:
an elongated component having a proximal end for facilitating handling and a distal end with an integrally formed echogenic portion for opacity during the ultrasound imaging process, the distal end for receiving and retaining at least one embryo; and
a cannula defining a channel for receiving the elongated component.

14. A catheter assembly as recited in claim 13, further comprising at least one mark on the elongated component for ascertaining a relationship between the distal end and the cannula.

15. A method for forming an echogenic end of an instrument comprising the steps of:
extruding a component having a distal end;
providing a die defining a cavity with a closed end;
heating the die; and
forming an echogenic portion of the distal end by compressing the distal end in the heated die.

16. A method as recited in claim 15, further comprising the step of removing the distal end from the die.

17. A method as recited in claim 15, further comprising the step of providing a central pin fixed within the cavity to maintain a passage in the distal end.

18. A method as recited in claim 15, wherein the instrument is polyurethane.

19. A method as recited in claim 15, wherein compressing the distal end increases a density of the component.

20. A method as recited in claim 15, wherein compressing the distal end increases a thickness of the component.

21. A method for implanting an embryo comprising the steps of:
providing a component for retaining an embryo at a distal tip thereof, the component having at least one marking at a predetermined distance from the distal tip;
providing a guide cannula for receiving the component such that the at least one marking indicates a position of the distal tip with respect to a distal end of the guide cannula;
placing at least one embryo on the distal tip of the component;
inserting the component into the guide cannula based upon the at least one marking such that the distal tip is covered by the distal end;
advancing the guide cannula and component into as os of a cervix of a patient;
advancing the component within the guide cannula such that the distal tip passes through the cervix and into a uterus of the patient;
implanting the embryo into the uterus; and
removing the guide cannula and component from the patient.

22. A method as recited in claim 21, wherein the distal tip is echogenic and further comprising the step of ultrasonically imaging the distal tip while inserted within the os of the cervix.
